(19) United States
(12) Patent Application Publication
(10) Pub. No.: US 2016/0029734 A1
(43) Pub. Date: Feb. 4, 2016

(54) FACEMASK AND FACEMASK SYSTEM

(71) Applicant: Snugmug LLC, Morro Bay, CA (US)

(72) Inventor: Kim Andre' Kurpjuweit, Morro Bay, CA (US)

(21) Appl. No.: 14/813,706

(22) Filed: Jul. 30, 2015

Related U.S. Application Data
(60) Provisional application No. 62/030,770, filed on Jul. 30, 2014.
(60) Provisional application No. 62/081,684, filed on Nov. 19, 2014.

(51) Int. Cl. A42B 3/20; A42B 3/18; A63B 71/10; A61F 9/02

(52) U.S. Cl. CPC A42B 3/205; A61F 9/02; A42B 3/185; A63B 71/10

(57) ABSTRACT

A facemask for use on a user's face includes a substantially rigid body portion having side edges on either side thereof, arms extending rearwardly from each of the side edges of the body portion, and connectors coupled to the arms. The facemask is configured to be positioned over the bottom part of a user's face in order to cover the cheeks, chin, and/or nose of the user. The connectors are coupled to the arms in order to mount each of the arms onto a mount and to hold the body portion in position on a user's face. The connectors are removable from a mount upon the application of a predetermined level of force and replaceable on a mount.

### CROSS-REFERENCE TO RELATED APPLICATIONS

This application claims priority to U.S. Provisional Application No. 62/030,770, filed Jul. 30, 2014 and U.S. Provisional Application No. 62/081,684, filed on Nov. 19, 2014, the disclosures of which are incorporated by reference in their entirety.

### FIELD

The technology described herein relates generally to a facemask, such as a facemask that can couple to a helmet, goggles 20 or other structure on the head of a wearer. In addition, the technology described herein concerns a mask for use with a helmet or goggles.

### BACKGROUND

Helmets or hats are often worn in sports to protect a participant's head or to keep the participant's head warm. In the field of skiing, skiers often wear helmets, hats, and/or goggles. The goggles are used to keep the sun, wind, and snow out of a skier's eyes. Goggles include an elastic strap that goes around a user's head, helmet, or hat. Modern ski helmets incorporate padding and other components that essentially replace a hat and help to keep a wearer's head warm. Many ski helmets, other than those worn for racing, typically have an open face. Hats and goggles also leave much of the face of a wearer open. In low temperature conditions, skiers often wear facemasks in order to keep their noses and chins warm. Facemasks may be fabric bandanas or neoprene facemasks, among other types of materials and configurations. Fabric and neoprene facemasks can keep a wearer's face warm for a limited period of time, but ultimately they become wet due to a skier's respiration. When wet, they can freeze and become cold to a user's face.

### SUMMARY

An example facemask system is shown and described. An example facemask can be used with a helmet or goggles, as shown and described.

### BRIEF DESCRIPTION OF THE DRAWING FIGURES

FIG. 1 is a perspective view of an example facemask according to the invention;

FIG. 2a is a schematic of a facemask and goggles according to the invention coupled to a helmet;

FIG. 2b is an expanded schematic of the facemask and visor of FIG. 2a, along with a helmet;

FIG. 2c is a schematic of a user wearing the facemask according to the invention, with the facemask being coupled to the strap of goggles;

FIG. 3 is front view of an example facemask;

FIG. 4 is a side view of an example facemask, with the opposite side being a mirror image thereof;

FIG. 5 is a front view of an alternative embodiment of the example facemask;

FIG. 6A depicts a side view of a mounting member of clip for use with a user's goggles in order to couple the facemask of the invention to the goggles on a user's head;

FIG. 6B depicts a front view of the clip of FIG. 6A;

FIG. 6C depicts a rear view of the clip of FIG. 6A;

FIG. 6D depicts a cross-sectional view through the cavity of the clip taken at line D-D in FIG. 6B;

FIG. 6E depicts an exploded cross-sectional view of one end of the cavity of the clip of FIG. 6D;

FIG. 7A depicts a plan view of the stainless steel or metallic member that is molded into the facemask in order to couple with magnets installed on the mount of FIGS. 6A-6E;

FIG. 7B depicts a side view of the metallic member of FIG. 7A;

FIG. 8 depicts a magnet that can be overmolded into the cavity of FIG. 6B;

FIG. 9 depicts two magnets being overmolded into the cavity in the arm of the facemask;

FIG. 10 depicts an alternative embodiment of the clip of the facemask, showing the clip being joined to the cavity of the clip, such as that in FIGS. 6A-6E, with only one magnet molded into the facemask 10 and either a magnet or a metallic member molded into the mounting member;

### DETAILED DESCRIPTION

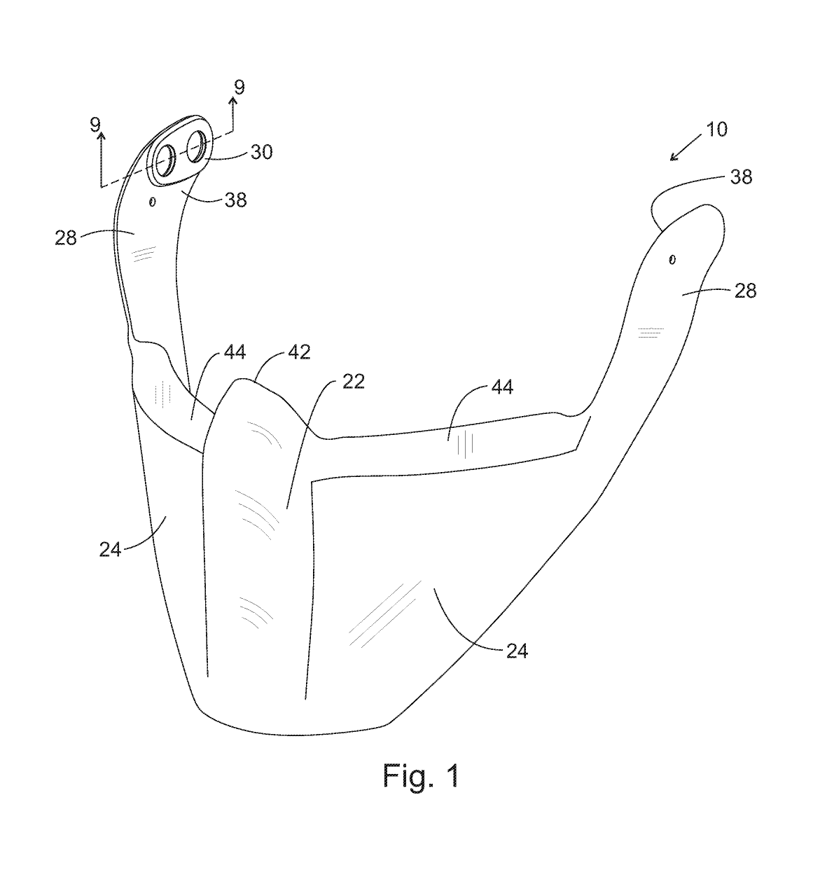

Referring to FIGS. 1-4, an example facemask 10 is provided for use in covering the face of a user in order to protect the face from the elements, such as cold, wind, snow, and rain, among other elements. The example facemask 10 secures to a user's head via a helmet, goggles, hat or other means, such as an independent strap. The facemask 10 is made of a material that is not prone to being affected by the elements. For example, with the prior art fabric and neoprene masks, they were prone to becoming wetted by a user's breath. The example facemask 10 forms to a user's face and maintains its shape. It does not become wet from respiration or perspiration. The facemask 10 can be used over and over again without getting wet and is easily washable with soap and water after use. The facemask 10 can be wiped down at any time and be returned to its substantially original condition. The facemask material is air impermeable and helps to keep a user's face warm because it does not permit air to travel through the facemask 10, except at particular locations where vents are provided. If vents are provided, they typically do not blow air onto a user's face and only provide venting for a user's breath.

The facemask 10 is provided in order to permit a removable and replaceable member for attaching to a helmet, a user's goggles, a hat or strap, or another piece of hardware worn on the head. The replaceable member includes a facemask that covers a portion of a user's face, including the nose, chin and cheeks, although it could cover less than the nose/chin/cheeks, or more. The facemask 10 typically does not include a portion that covers the eyes. As shown in FIG. 1, the facemask 10 extends above the nose and under the eyes of a user. Although not shown, in one embodiment, the facemask 10 could include a part that covers or shields the eyes. A separate replaceable member, such as a visor 12, may also be included in the system. The facemask 10 and visor 12 can be removably coupled to a supporting structure or member 14, such as a surface of a helmet 16, to the strap 18 of a pair of goggles 20, to a hat (not shown), or to another member on the head (not shown). The facemask 10/visor 12 can be coupled and uncoupled to the supporting structure 14 as needed and otherwise stored away from the supporting structure 14.

For example, the facemask 10 may be used during cold weather in order to protect a user's face from direct exposure to the elements. The visor 12 may be used when it is sunny or snowing in order to shield the eyes from sun and snow. Both the visor 12 and the facemask 10 may be removed from the supporting structure 14 when desired.

Referring to FIG. 1, a facemask 10 is shown that generally conforms to the shape of a user's face. The facemask 10 includes a nose portion 22 that extends outwardly from a main wall 24 of the facemask 10. The main wall 24 of the facemask 10 covers the chin and cheeks of a user. The facemask 10 may angle inwardly toward the face at an upper end 26 that would typically be directly below the eyes. The nose portion 22, as shown, extends upwardly in order to cover the upper part of the nose, between the eyes. The facemask 10 may cover as much of the face as possible below the eyes. As mentioned above, although not shown in FIG. 1, the facemask 10 could include a portion that covers the eyes and all or part of the forehead of a user, as well as part of the neck and sides of the head, including ears (not shown). Typically, when a helmet 16 or hat is worn by a user, the helmet 16 or hat will cover the ears, head, and majority of the forehead. As such, the example shown in FIG. 1 is meant to cover the remainder of the face, other than the eyes. Skiers often wear goggles 20 or sunglasses under their helmets 16. The goggles 20 will cover the eyes of the user with the facemask 10 being fitted directly under the goggles. The facemask 10 works with the goggles 20 to provide a large amount of protection to the face of the user.

As shown in FIG. 1, the facemask 10 includes arms 28 that extend rearwardly and/or upwardly. When worn, the facemask arms 28 extend towards the wearer's temples. The arms 28 have an inwardly facing attachment feature 30 that can couple with a supporting structure 14 in order to removably couple the facemask 10 to a helmet 16, goggles 20, hat, or other mechanism worn on the head. For example, a separate strap that extends around the head could be provided if a goggles 20, helmet 16, or hat is not worn. The attachment mechanism 30 shown herein is magnetic. Other types of attachment mechanisms 30 could also be used, such as snaps, Velcro, and the like. The attachment mechanisms are also referred to herein as connectors 30.

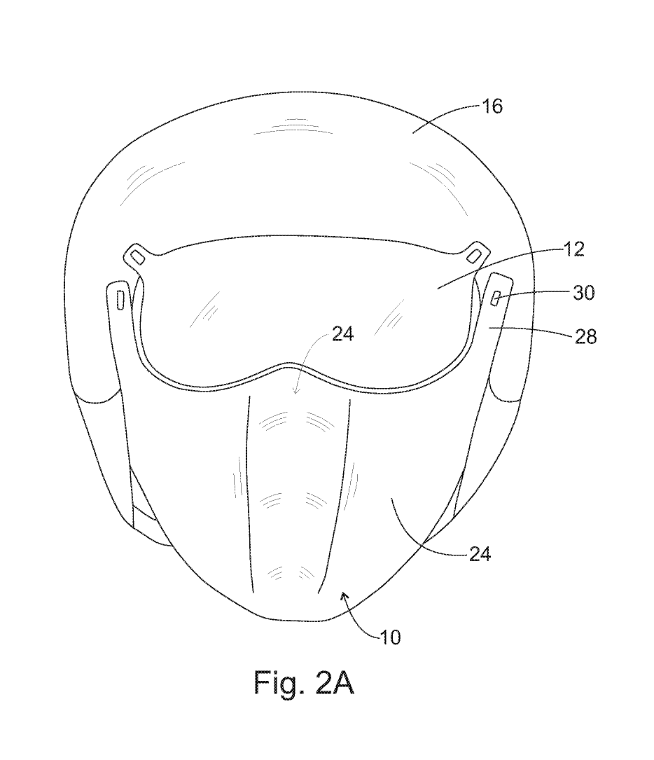

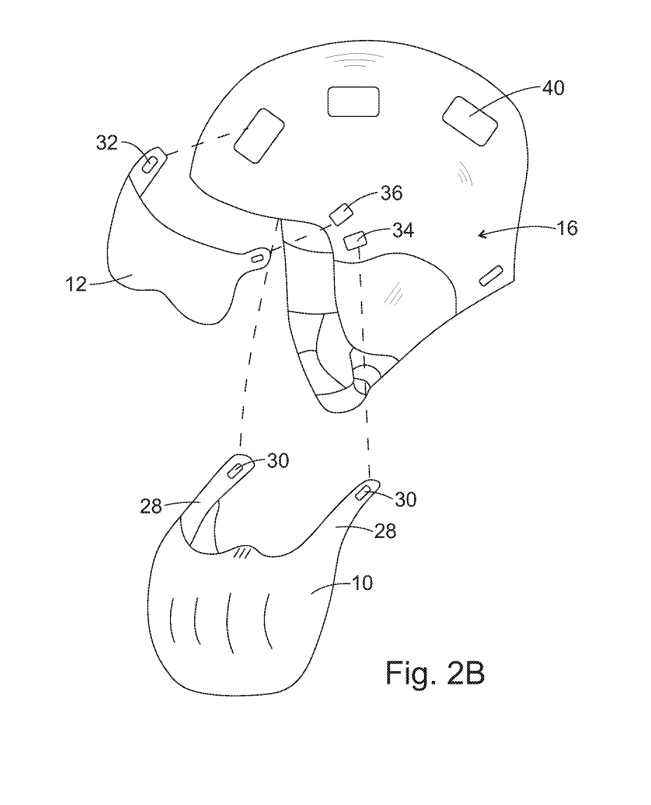

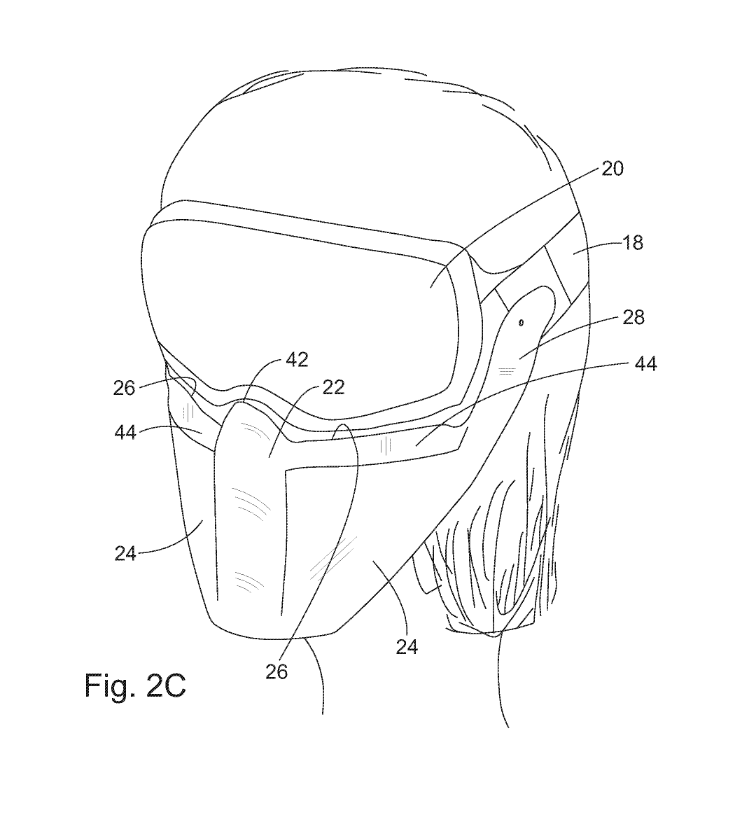

FIGS. 2A-2C depict a facemask 10 and visor 12 according to the invention installed on a helmet 16 and on a strap 18 of goggles 20. As shown in FIG. 2A, the user is wearing a helmet 16 and has a facemask 10 coupled to the helmet 16 such that it covers the nose, chin and cheeks of the user's face. The user is also wearing a visor 12 that couples to the helmet 16 such that most of the face of the user is protected from the elements. If desired, the visor 12 and facemask 10 could be integrated together. The visor 12 could be made prescription, if needed. The facemask 10 has upwardly extending arms 28 that extend toward the temples of a user, where they connect with a supporting structure 14 on the helmet 16. Likewise, the visor 12 includes arms 32 that extend toward the user's temples and connect with a supporting structure 14 on the helmet 16. The arms 28 could be configured so that the facemask 10 mates with a supporting structure 14 on the helmet 16 or goggles 20 and the visor 12 mates on top of the arm 28 of the facemask 10.

Two different mounts 34, 36 are shown as being positioned on an outer surface of each side of the helmet 16. An upper mount 36 is used for the visor 12 and a lower mount 34 is used for the facemask 10. As will be explained in greater detail below, a single mount 34 may also be used, so that an upper and a lower mount are not both needed.

FIG. 2B shows an exploded view of the helmet 16 with facemask 10 and visor 12, showing that separate supporting structures 14 or mounts 34 are provided on the surface of the helmet 16. The supporting structures 14 can be positioned on the helmet 16 in any known manner, such as by coupling the supporting structures 14 to the helmet 16 during the manufacturing process so that they are permanently installed on the helmet 16, or by gluing or using hook and loop tape that is adhered to the helmet 16, among other techniques for attaching them (not shown).

FIG. 2C is another view of the example facemask 10 installed on a user's face when the user is wearing goggles 20. The goggles 20 have a strap 18 that goes around a user's head. Even if a user were wearing a helmet 16, the strap 18 of the goggles 20 would go around the helmet 16 and, thus, the user's head. The embodiment shown in FIG. 2C permits the user to couple the facemask 10 to a strap 18 of the goggles 20 such that the arms 28 of the facemask 10 mate with a connector 30 on the straps 18 in the vicinity of the temples of the wearer's head. As with the prior embodiment, the user's chin, cheeks and nose are covered by the facemask 10 while the goggles 20 cover the user's eyes. A supporting structure 14 is positioned on the strap 18 of the goggles 20 in order to couple the attachment mechanism 30 on the arms of the facemask 10 to the supporting member 14 on the goggles 20. The supporting member 14 may be integral with the goggles 20 or goggle straps 18, or may be added to the goggles 20 by the user. For example, the facemask 10 may come with a supporting member 14 to be added to the goggles 20.

The facemask 10 is typically made of a hard material, such as a hard opaque or clear material. The helmet 16 that is utilized with the facemask 10 is a standard, off the shelf helmet 16. The visor 12 may be made of a clear plastic material. The visor 12 may be tinted. The goggles 20 utilized with the facemask 10 may be standard over-the-counter goggles 20, or may be prescription or specialized goggles.

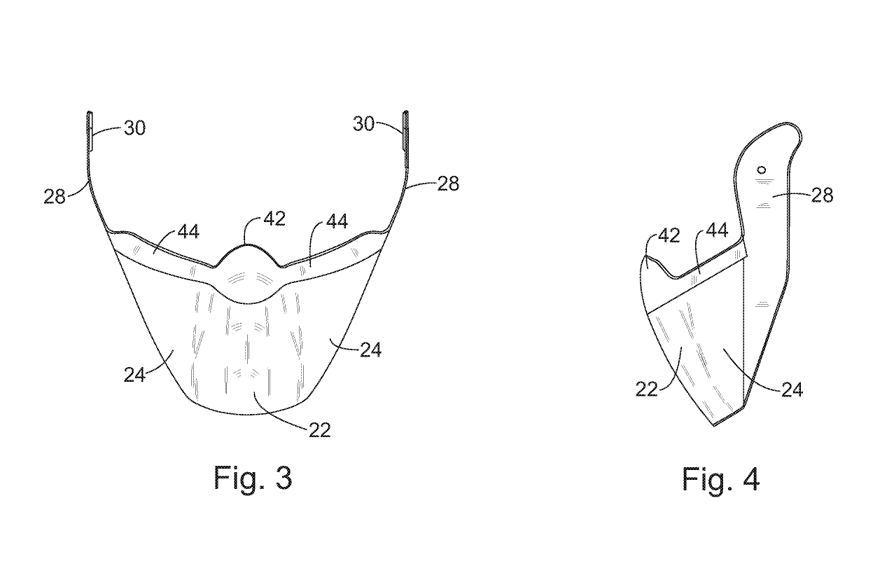

Referring to FIGS. 3 and 4, an example facemask 10 is shown from a front view and a side view, with the opposite side being a mirror image thereof. The facemask 10 includes a main wall 24 and nose portion 22 for covering a significant portion of the user's face, such as the lower portion of the face that is below the nose, or that includes coverage for the nose. The facemask 10 may be contoured to fit the chin and face, or could be less contoured, if desired. The facemask 10 includes attachment arms 28 that have connectors 30 that permit the arms 28 to be coupled to a supporting structure 14 having mounts for mating with the connectors 30. The arms 28 extend so that an inner surface 38 of the arms 28 is substantially parallel to an outer surface of the user's head or helmet 16 when the user is wearing a helmet 16. The connectors 30 are positioned on an inner surface 38 of the arms 28. The facemask 10 includes a portion 22 that extends over the nose so that the entire chin, mouth and nose can be covered, if desired. The facemask 10 and/or visor 12 may include apertures, such as vents 40, therethrough that permit air to enter and exit the facemask 10.

The facemask 10 may include a logo (not shown) that is positioned on a side surface of the facemask 10. The logo may be used for advertising and for brand identification, among other known uses. The facemask 10 may also include instructions (not shown) for coupling the facemask 10 to a helmet 16 or a supporting structure 14, if desired. When a visor 12 is used with the system, the visor 12 may also have a logo or other branding positioned on an outer surface of the visor 12. The logo may be positioned at any position on the facemask 10 or visor 12, as desired, including on the front or back of the mask. Alternatively, a logo is not required to be placed on a surface of the facemask 10 or visor 12.

In addition to the logo or instead of the logo, the mask may include images and/or designs in order to change the outward appearance of the mask from its original material. For example, the mask may have artwork, designs, names, and other images on the surface of the mask. In one embodiment, the mask is hydro-dipped in order to provide an image or pattern on the surface of the mask. Alternatively, different colored materials may be used as a base material for the mask so that the mask takes on the color of the material or materials. Materials could be swirled or otherwise altered from a plain color. Patterns could be applied to the mask using painting, wraps, etching, or other techniques, as known by those of skill in the art. Any type of adornment may be used that can be applied to a plastic material.

The supporting structure 14 includes at least a portion that is a mount 34 for coupling the facemask 10 to the user's head in a semi-permanent manner. For example, the supporting structure 14 can be provided on the side surfaces of a helmet 16, on slides positioned on a strap 18 of a goggles 20, on a hat, or on a strap that extends around the back of a user's head. When installed on a helmet 16, the supporting structure 14 can be coupled using a semi-permanent or a permanent tape. One type of tape that may be used is 3M VHB foam PE tape. Alternatively, the supporting structure 14 could be glued, welded, screwed, or molded into the helmet 16, if desired.

The attachment arms 28 of the facemask 10 are configured to mate with the mounts 34 of the supporting structures 14 that are fixed to the helmet 16. The ends of the attachment arms 28 face inwardly and the connectors 30 are positioned on the inner side 38 of the arms 28 for mating with the mounts 34 on the supporting structure 14. The mounts 34 on the supporting structure 14 face outwardly, away from the sides of the user's head. The connectors 30 and mounts 34 on the supporting structures 14 have components that are configured to mate in a fixed relation. One type of mating component is mating magnets, with one magnet being positioned on the supporting structure 14 mount and a mating magnet being positioned on an inner surface 38 of the attachment arms 28. Instead of mating magnets, one of the surfaces may include a magnet and one of the surfaces may include or be a metal material. Other types of mounts may be used, such as hook and look tape, snaps, and other attachment means.

The facemask 10 shown in FIGS. 3 and 4 has a central upwardly extending flange 42 that extends over the user's nose. The facemask 10 extends downwardly from the nose flange 42 and provides space between the facemask 10 and the user's lips and mouth. The facemask 10 may be contoured to fit the chin and face, or could be less contoured, if desired. The front sides of the facemask 10 closely conform to the contours of the cheeks. In addition, an upper ledge 44 of the facemask 10 that is positioned below the user's eyes angles inwardly in order to deter air flow under the facemask 10 around the eyes of the user. The facemask 10 includes attachment arms 28 that extend rearwardly and upwardly from the side edges of the facemask 10 so that an inner surface 38 of the arms 28 is substantially parallel to an outer surface of the user's head. The connectors 30 are positioned at the ends of the arms 28 on an inner surface 38 thereof. The facemask 10 may include apertures 40 therethrough that permit air to enter and exit the facemask 10, although the embodiment shown in FIGS. 3 and 4 does not include such apertures.

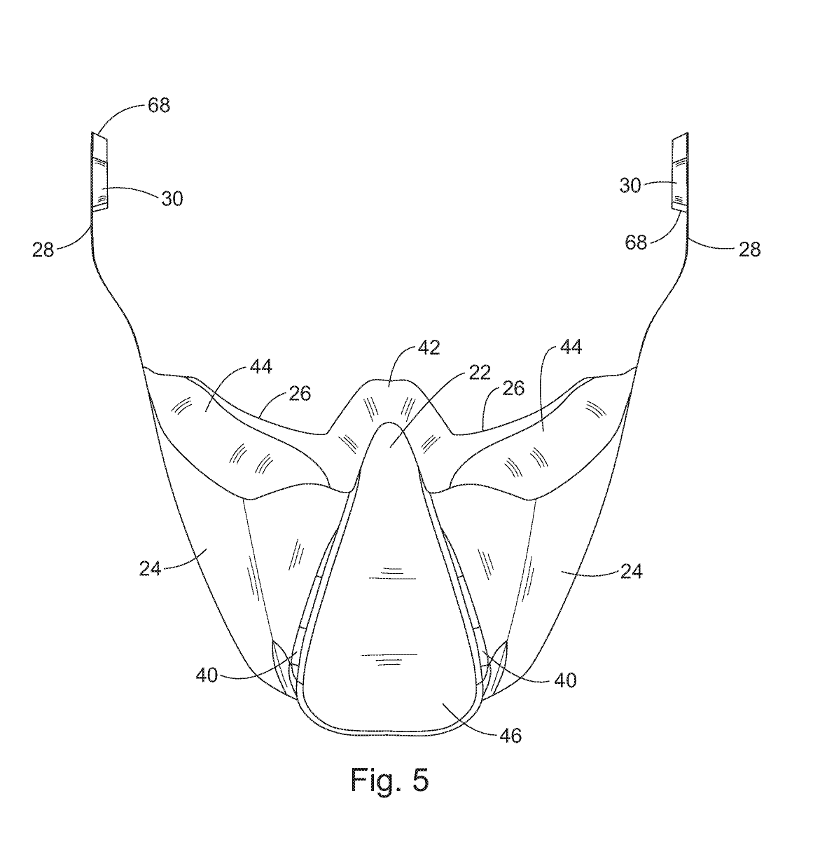

FIG. 5 depicts a front view of an alternative facemask 10 that has a flat front area 46 in the vicinity of the nose and mouth, with vents 40 on the sides of the front area 46. In addition, the facemask 10 has a dual contoured surface in the area of the facemask 10 that is positioned below a user's eyes and over a user's nose. The facemask 10 includes an elongated, triangular central portion 46 that extends forward of the nose and mouth. Then the cheek area of the facemask 10 extends rearwardly and outwardly from the central triangular portion 46. The attachment arms 28 have a curvature that permits them to be farther away from the user's head. This design is advantageous if the user is wearing a helmet 16 instead of a hat or goggles 20, since the ends of the arms 28 can fit around the sides of a helmet 16. As with the prior design, connectors 30 are positioned on an inner surface 38 of the arms 28 and face inwardly such that are substantially parallel to the side of a user's head. When the facemask 10 is installed on a user's head, the connectors 30 are positioned in the vicinity of the user's temples. The underside of the facemask 10, although not shown, may be smooth in order to prevent any scratching of the user's face. The underside of the facemask 10 could be coated with a smooth polymeric material, such as silicone, which is smooth and soft, but does not collect water. The facemask 10 can be made of ABS/PC Cycoloy 1200HF or another material, if desired.

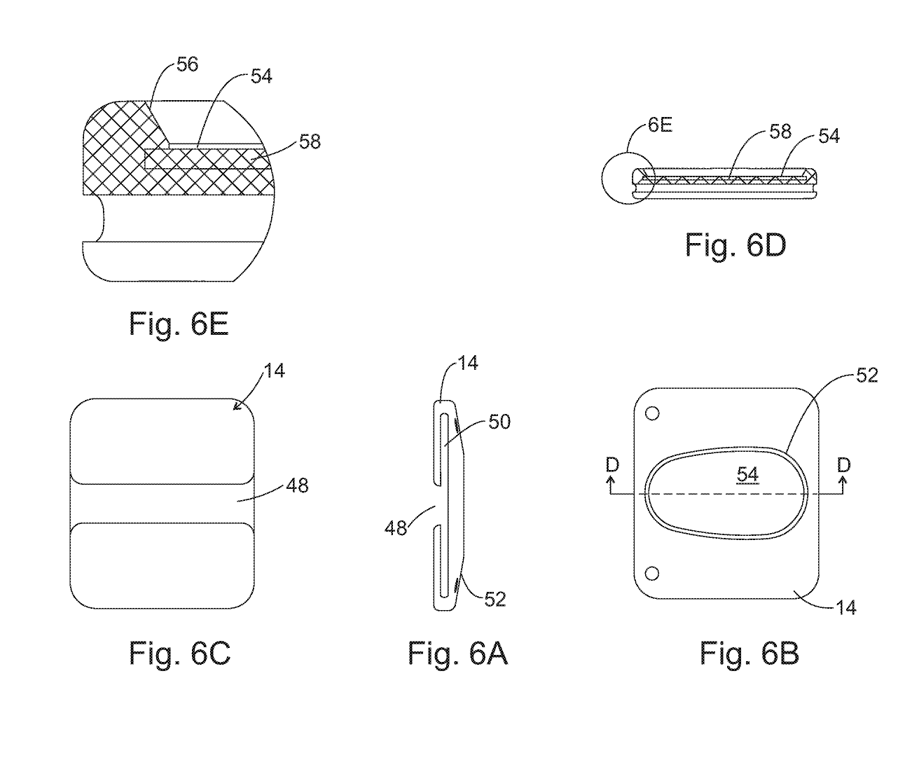

FIGS. 6A-6E show an example supporting structure 14 that can be used on a goggle strap 18 in order to serve as a mount 34 for the attachment arms 28. Two supporting structures 14 would be used, with one being positioned on either side of a user's head. This design could be modified to be positioned on a helmet 16, coupled to a hat, or coupled to a strap that extends around the head. The example shown in FIGS. 6A-6E is a slider that is to be positioned around a strap 18, such as a goggle strap 18.

FIG. 6A shows a side view of the supporting structure 14, with a mount 34 being located on a front side of the structure and a slot opening 48 and recess 50 on a rear side of the supporting structure 14 for enclosing a strap 18. The strap 18 of the goggles 20 extends through the slot opening 48 and then the strap 18 opens into the recess 50 so that the supporting structure 14 can slide on the strap 18 in the recess 50. The mount 34 is better shown in the front view of FIG. 6B and includes a raised area 52 that is ovoid or egg-shaped that surrounds a depression 54. The depression 54 is for receiving the raised portion of the connector 30 of the attachment arm 28. The outwardly facing surface first slopes upwardly and then slopes downwardly and inwardly until it meets the inner surface of the depression 54. The inner sloped wall 56 is angled such that it is conducive to permitting the attachment arm connector 30 to slide into the depression 54. For example, the sloped wall 56 could be angled at a 30 degree angle.

The front view in FIG. 6B represents the outwardly facing portion of the supporting structure 14 that is configured to join with the connector 30 on the attachment arms 28 of the facemask 10. The ovoid area of the mount is used to hold either a magnet or a metallic member, such as a stainless steel member 58. The metallic member 58 may be positioned and fixed in the depression 54 of the mount 34 or may be molded into the depression 54 of the mount 34 during the manufacturing process so that the metallic member 58 is not exposed.

FIG. 6C shows a rear view of the supporting structure 14, which has the slot 48 for permitting the entry of the strap 18 into the recess 50 in the supporting structure 14. This part of the supporting structure 14 is optional if the supporting structure 14 is not to be mounted on a strap 18. For example, the supporting structure 14 could be sewn, riveted, or otherwise attached to the sides of a hat, in which case the slot 48 and recess 50 would not be needed. Alternatively, the supporting structure 14 could be adhered to the side of a helmet 16 using an adhesive, rivets, hook and look tape and other attachment means, in which case the slot 48 and recess 50 would not be needed. Alternatively, the supporting structure 14 could be integrally formed into the shell of a helmet 16, such that the slot 48 and recess 50 on the rear side of the supporting structure 14 would not be needed and only the portion of the supporting structure 14 shown in FIG. 6B would be needed.

FIG. 6D shows a cross-sectional view of the ovoid area of FIG. 6B, taken at line D-D in FIG. 6B. FIG. 6E depicts an expanded view of the left end of the cross-section n FIG. 6D. As previously discussed, the ovoid area serves as the mount 34 for the connector 30 of the attachment arms 28 and defines a recess 54 into which the connector 30 can be guided. FIGS. 6D and 6E show the inwardly sloped wall 56 of the recess 54, as well as the location of the metallic member 58 inside the wall 56 of the recess 54. The metallic member 58 can be overmolded so that it is completely covered with a thin layer of plastic material, or can be overmolded so that an upper surface of the metallic member 58 is still visible in the depression 54, but the ends of the metallic member 58 are trapped by the material of the supporting structure 14. An ovoid shape is shown, but other shapes could be used. The mount can be made of ABS/PC Cycoloy 1200HF, if desired, or of a different material.

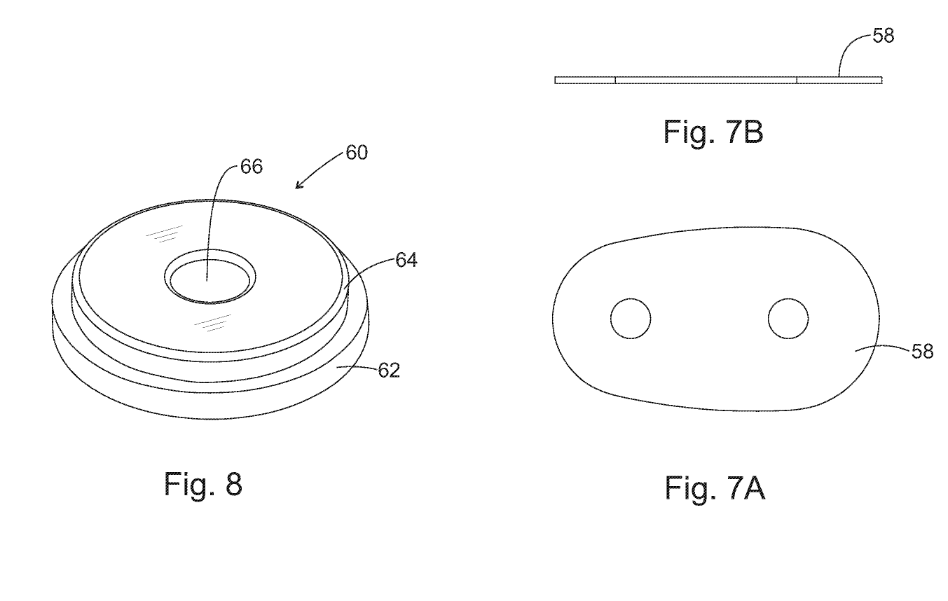

FIGS. 7A & 7B depict a metallic insert 58 that can be used in the mount of FIGS. 6A-6E. FIG. 7A depicts a front view showing that the metallic insert 58 can be ovoid shaped, like the shape of the mount 34. Alternatively, the metallic insert 58 could be a shape that is different from the shape of the mount 34. FIG. 7B depicts a side view of the metallic insert 58, with the insert being thin and flat. The metallic insert 58 may have openings in the surface or extending therethrough. The openings may assist in permitting the material of the mount to follow around the insert when being overmolded, in order to properly secure the insert into the overmolded portion. If the mount 34 is a different shape, the metallic insert 58 could be the same shape as the mount or a different shape. As previously discussed, the insert 58 could be a metallic member or could be a magnet for mating with the magnet in the connector 30 on the attachment arm 28, with the magnets being of reverse polarity so they are capable of coupling together.

FIGS. 8-10 depict the attachment mechanisms or connectors 30 on the ends of the attachment arms 28. FIG. 8 depicts a magnet 60 that can be used in the attachment arms 28. This magnet 60 can be overmolded into the facemask 10 arm during the manufacturing process. Alternatively, this magnet 60 or a different magnet could be coupled to the attachment arms 28 in any known manner. The magnet 60 shown in FIG. 8 has a round periphery that is stepped from a larger diameter at a lower end 62 to a smaller diameter at an upper end 64. The magnet 60 is also shown as having a circular recess 66 in the center of the upper end 64. The top and bottom surfaces of the magnet are substantially flat.

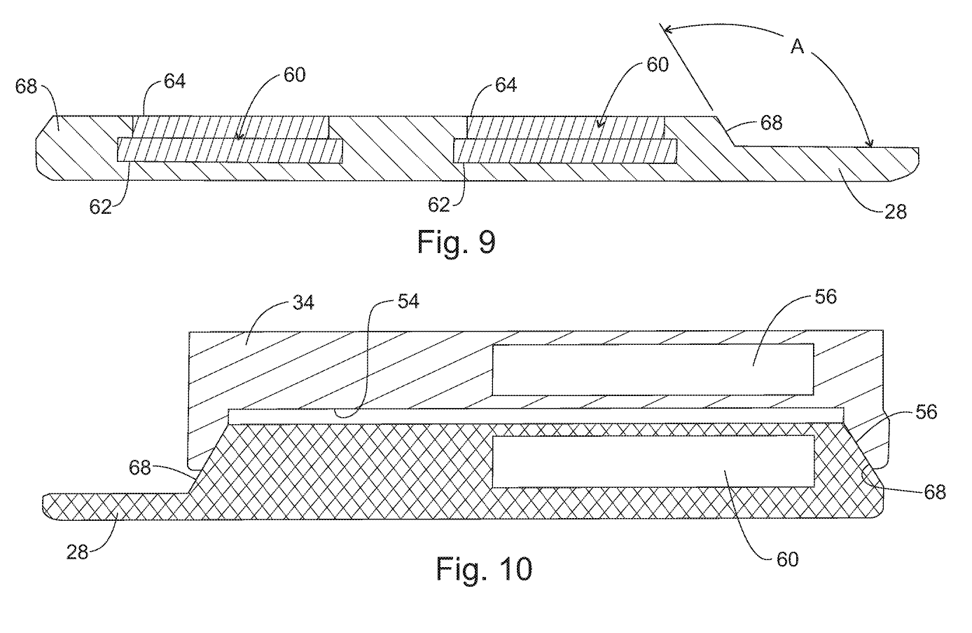

FIG. 9 depicts a connector 30 of the attachment arm 28, taken from the cross-section in FIG. 1. In this embodiment, the connector 30 has two magnets 60 embedded in the material of the arm 28. The magnets 60 may be embedded during the manufacturing process by overmolding or other known means. The larger diameter bottom portion 62 is entirely enclosed in the material of the arm 28 while the smaller diameter upper end 64 of the magnet 60 is exposed at an inner side 38 of the arm 28. The connector 30 has sloped surfaces 68 around the sides of the connector 30 for mating with the sloped surfaces 56 of the depression 54 in the mount 34. For example, the slope of the side surface 68 may be approximately 120 degrees. This slope 68 can be on all sides of the connector 30 in order to permit the connector 30 to be guided into the recess 54 of the mount 34, although not absolutely required.

FIG. 10 shows how the connector 30 and mount 34 can couple together, but for an alternative embodiment where the connector only includes one magnet 60 and the mount only includes one magnet or metallic member 58. This example shows how the sloped side walls 58 of the recess 54 help to guide the connector 30 into the recess 54 such that the side surfaces 58, 68 mate. The walls 58, 68 are angled and the protrusion of the connector 30 is sized so that the face of the connector 30 does not engage the face of the recess 54 of the mount 34. In this example, the magnets/metallic members 60, 58 are entirely encapsulated within the surrounding material. Alternatively, the magnets could be exposed.

While not shown, the magnet 60 may include a retaining band around it in order to allow for overmolding during the manufacturing process. Any known technique for overmolding or mounting the magnets or metallic members to the various parts may be utilized. The mount 54 may be a harder or stronger compound than that of the facemask 10, or the mount 54 may be the same type of material as the facemask 10. The mounts 34 on the supporting structure 14 may be magnets or metal parts. As metal parts, they may be, for example, 420 grade stainless steel, or other types of materials.

As shown in FIGS. 2A and 2B, a separate mount or connector may be coupled to the surface of the helmet 16 for receiving an attachment arm 32 of a visor 12, if desired. Alternatively, the attachment arms 28 of the facemask 10 may include an outwardly facing mount (not shown) for coupling with a connector that is positioned on an inner surface of the visor arms 32, utilizing the same techniques as were used for the mount on the supporting structure 14.

While two mounts 34 are shown and described herein, with the mounts being positioned on opposite sides of the helmet 16, a system could utilize a single mount, such as one where the mount is positioned above the opening for the face, and a facemask 10 extends downwardly over the face from the single mount (not shown).

The connector 30 could have other shapes, such as round, rectangular, oval, irregular, or the like, as long the connector is fashioned so that it properly couples to the mount. Likewise, the mount 34 may have any known shape.

Other types of mounts 34 and connector designs 30 can be utilized, the invention not being limited to a particular type of mount or connector. All that is required is that the connector arms 28 be maintained in a removably, fixed relation to the mount 34 when installed. For example, snaps, clips, or other mechanical connectors may be used. The connection between the connector 30 and the mount 34 must be strong enough to maintain the facemask 10 in position on the user's face under normal operating conditions. Other shapes for the facemask 10 and visor 12 can be used.

A leash (not shown) may be provided that couples the facemask 10 and/or visor 12 to the helmet 16, goggles 20, or another member so that the facemask 10 and/or visor 12 can be removed from the helmet 16 or goggles 20 and will hang from the side thereof when not needed, such as when the user is riding a lift or waiting in the lift line. The leash may be a fabric or cord leash and may be coupled to the helmet 16 or goggles 20 and to the facemask 10 in any known manner.

While not shown, the mounts 34 or connectors 30 may permit multiple layers of connections. For example, the facemask 10 may couple to the mount that is positioned on an outer surface of the helmet 16, or the visor 12 may couple to the mount that is positioned on an outer surface of the helmet 16. In addition, the facemask 10 may couple to the mount and the visor 12 may couple over the facemask 10 at the same mount location, with the facemask arms 28 having a like mount positioned on an outer surface of the arms 28 in order to permit the stacking of mounts 34 and mating with the connectors 30 on the visor 12. Thus, multiple mounts 34 may be stacked such that only a single mount location is needed. While magnetic mounts 34 are described herein˜other types of mounts 34 and connectors 30 may be utilized, a known by those of skill in the art, including snaps, hooks, or the like.

While the example facemask system presumes that the facemask 10 will be added to the helmet 16 or goggles 20 as an aftermarket upgrade, the facemask system could be incorporated in a helmet 16 during the manufacturing of the helmet 16. Likewise, the mount 34 could be incorporated in the goggles 20 during the manufacturing of the googles so that separate clips or support structures 14 are not needed. The helmet 16 could include a pair of integral mounts 34 positioned on an outer surface of the helmet 16 for mating with the facemask 10 that has connectors 30 configured to mate with the mounts 34. The facemask 10 could be sold with the helmet 16, or could be sold separately from the helmet 16. Multiple different types of facemasks 10 could be made available for purchase with a single helmet 16. The same is true for the visor 12. The helmet 16 could include integral mounts 36 for coupling to a visor 12. The mounts 36 could be separate from the mounts 34 for the facemask 10, or could be the same mounts 34 as are used for the facemask 10, with the facemask 10 and visor 12 being stackable at the mount/connector connection. The helmet 16 could be sold without the visor or with one type of visor, and additional visors could be made available for separate purchase.

The supporting structure 14 and facemask 10 may be made of a polymeric or plastic material, among other materials, including fiber or cloth materials, as long as the facemask 10 can maintain a fixed shape. Other types of materials may also be utilized, as known by those of skill in the art. The supporting structures 14 may be made of a material type that is different from the facemask 10. If desired, the facemask 10 and/or supporting structures 14may be made of multiple types of materials that are joined or layered together.

In one embodiment, a facemask 10 for use on a user's face includes a body portion, an arm and connectors 30. The body portion is substantially rigid and is configured to be positioned over the bottom part of a user's face in order to cover the cheeks, chin, and/or nose of the user. The body portion has side edges on either side of the body portion. An arm extends rearwardly from each of the side edges of the body portion. Connectors 30 are coupled to the arms 28 in order to mount each of the arms 28 onto a mount and to hold the body portion in position on a user's face. The connectors 30 are removable from a mount upon the application of a predetermined level of force and replaceable on a mount.

The body portion may be contoured to cover a user's chin, cheeks, and nose, but not the user's eyes. The arms 28 may have an inwardly facing surface at an end thereof that is substantially parallel to a side of the user's head. The connectors 30 may include a magnetic component. The body portion may be made of a rigid or semi-rigid polymeric material. The body portion may be made of a material that does not store water. The arms 28 may be integral with the body portion.

In another embodiment, a facemask system includes the facemask 10 described above and at least two mounts. The mounts 34 are configured to couple to connectors 30 of the attachment arms 28 in order to couple the facemask 10 to a user's head.

The mounts 34 may be coupled to a supporting structure 14 and the supporting structure 14 may be one or more of a slider for positioning on a strap 18 and having the mount coupled thereto, a stationary member for mounting to a helmet 16 and having the mount coupled thereto, an eye covering member having a portion extending around a user's head and having the mount coupled thereto, a strap 18 having the mount coupled thereto, and a hat having the mount coupled thereto.

They eye covering members may be goggles 20 that have a strap 18 that extends around a user's head. The supporting structure 14 may be positioned on a slider that slides upon the strap 18 of the goggles 20. The mount may have a metallic portion or a magnetic portion and the connector may be configured to mate with the mount. The connector may have a magnetic portion for mating with the metallic portion or the magnetic portion of the mount. The mount may include a recess for receiving a protrusion of the connector, with the recess having sloped side walls for introducing the connector into the recess. The mount may include a first mount that is positioned on one side of the user's face or head and a second mount that is positioned on the opposite side of the user's face or head. The facemask arms 28 mate with the first and second mounts. The mount may include a recess for receiving a metallic member or a magnetic member. The metallic or magnetic members may be encapsulated or overmolded into the mount. The connector may include a metallic member or a magnetic member that is encapsulated or overmolded into the arm. The mount may be a removable clip having a recess for capturing the strap 18 of the goggles 20 in the recess.

In yet another embodiment, a facemask 10 for use with goggles 20 or a helmet 16 of a user includes a facemask 10 having a body portion for covering at least a portion of a user's face, and arms 28 extending from the body portion. The arms 28 are for mating with a mount in order to couple the facemask 10 to one of goggles 20 or a helmet 16 of a user. The facemask 10 also includes a connector coupled to each of the arms 28 of the body portion. The connectors 30 are configured to mate with the mount. The facemask 10 is removable and replaceable on the mount and remains in position on the mount during normal use.

The connector may include a magnetic component and the mount may include a magnetic component or a metallic component. The facemask 10 remains removably fixed to the mount during normal use, such that a predetermined level of force is required in order to remove the facemask 10 from the mount. The goggles 20 may include a face covering portion and one or more straps 18 that extend around a user's head, with the mount being coupled to the face covering portion or to the one or more straps 18. The mount may be coupled to a supporting structure 14 that has a recess for receiving the one or more straps 18.

The term “substantially” is used herein as a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What is claimed is:

1. A facemask for use on a user's face comprising:
a substantially rigid body portion configured to be positioned over the bottom part of a user's face in order to cover the cheeks, chin, and/or nose of the user, said body portion having side edges on either side of the body portion;
an arm extends rearwardly from each of the side edges of the body portion; and
connectors coupled to the arms in order to mount each of the arms onto a mount and to hold the body portion in position on a user's face, with the connectors being removable from a mount upon the application of a predetermined level of force and replaceable on a mount.

2. The facemask of claim 1, wherein the body portion is contoured to cover a user's chin, cheeks, and nose, but not the user's eyes.

3. The facemask of claim 1, wherein the arms have an inwardly facing surface at an end thereof that is substantially parallel to a side of the user's head.

4. The facemask of claim 1, wherein the connectors includes a magnetic component.

5. The facemask of claim 1, wherein the body portion is made of a rigid or semi-rigid polymeric material, and the body portion is made of a material that does not store water.

6. The facemask of claim 1, wherein the arms are integral with the body portion.

7. A facemask system comprising:
the facemask of claim 1; and
at least two mounts, with the mounts being configured to couple to connectors of the attachment arms in order to couple the facemask to a user's head.

8. The facemask system of claim 7, wherein the mounts are coupled to a supporting structure; and the supporting structure is one or more of:
a slider for positioning on a strap and having the mount coupled thereto;
a stationary member for mounting to a helmet and having the mount coupled thereto;
an eye covering member having a portion extending around a user's head and having the mount coupled thereto;
a strap having the mount coupled thereto; and
a hat having the mount coupled thereto.

9. The facemask system of claim 8, wherein the eye covering member is goggles that have a strap that extends around a user's head, and the supporting structure is positioned on a slider that slides upon the strap of the goggles.

10. The facemask system of claim 8, wherein the mount has a metallic portion or a magnetic portion and the connector is configured to mate with the mount.

11. The facemask system of claim 10, wherein the connector has a magnetic portion for mating with the metallic portion or the magnetic portion of the mount.

12. The facemask system of claim 10, wherein the mount includes a recess for receiving a protrusion of the connector, with the recess having sloped side walls for introducing the connector into the recess.

13. The facemask system of claim 8, wherein the mount includes a first mount that is positioned on one side of the user's face or head and a second mount that is positioned on the opposite side of the user's face or head, and the facemask arms mate with the first and second mounts.

14. The facemask system of claim 8, wherein the mount includes a recess for receiving a metallic member or a magnetic member, and the metallic or magnetic members are encapsulated or overmolded into the mount, and the connector includes a metallic member or a magnetic member that is encapsulated or overmolded into the arm.

15. The facemask system of claim 9, wherein the mount is a removable clip having a recess for capturing the strap of the goggles in the recess.

16. A facemask for use with goggles or a helmet of a user comprising:
a facemask having a body portion for covering at least a portion of a user's face, and arms extending from the body portion, said arms for mating with a mount in order to couple the facemask to one of goggles or a helmet of a user; and
a connector coupled to each of the arms of the body portion, said connectors being configured to mate with the mount,
wherein the facemask is removable and replaceable on the mount and remains in position on the mount during normal use.

17. The mask of claim 16, wherein the connector includes a magnetic component and the mount includes a magnetic component or a metallic component.

18. The mask of claim 16, wherein the facemask remains removably fixed to the mount during normal use, such that a predetermined level of force is required in order to remove the facemask from the mount.

19. The mask of claim 16, wherein the goggles include a face covering portion and one or more straps that extend around a user's head, with the mount being coupled to the face covering portion or to the one or more straps.

20. The mask of claim 19, wherein the mount is coupled to a supporting structure that has a recess for receiving the one or more straps.
